(19) United States
(12) Patent Application Publication
(10) Pub. No.: US 2024/0065886 A1
(43) Pub. Date: Feb. 29, 2024

(54) NOZZLE FOR A FLUID DELIVERY DEVICE

(71) Applicant: TWENTY TWENTY THERAPEUTICS LLC, South San Francisco, CA (US)

(72) Inventor: Timothy Stowe, Alameda, CA (US)

(21) Appl. No.: 18/238,432

(22) Filed: Aug. 25, 2023

Related U.S. Application Data
(60) Provisional application No. 63/400,933, filed on Aug. 25, 2022.

(51) Int. Cl. A61F 9/00

(52) U.S. Cl. CPC A61F 9/0008

(57) ABSTRACT

A non-gravitational fluid delivery device is provided for delivering fluid to an eye of a user. The device includes a nozzle having a nozzle wall. The nozzle wall has opposing interior and exterior nozzle surfaces, and a plurality of openings dispersed along a longitudinal nozzle width of the nozzle wall through which fluid is configured to be selectively delivered to the eye during use of the device. Each opening extends through the nozzle wall from a substantially rectangular entry port in the interior nozzle surface to a substantially rectangular port in the exterior nozzle surface. Each of the openings has a longitudinal opening width that is less than a lateral opening length.

### RELATED APPLICATION

This application claims priority from U.S. Provisional Application No. 63/400,933, filed 25 Aug. 2022, the subject matter of which is incorporated herein by reference in its entirety.

### TECHNICAL FIELD

This disclosure relates to a nozzle for a fluid delivery device and a non-gravitational fluid delivery device for delivering fluid to an eye of a user.

### BACKGROUND

Non-gravitational fluid delivery devices for the non-gravitational delivery of fluids (e.g., ophthalmic drugs and/or viscous ophthalmic drugs) to a target site of the user (e.g., to the user's eye(s), nose, and/or mouth) are known. For example, U.S. patent application Ser. No. 15/931,482 (“the '482 application”), filed 13 May 2020 by Stowe and titled “Non-Gravitational Fluid Delivery Device For Ophthalmic Applications”, the subject matter of which is incorporated by reference in its entirety, discloses such non-gravitational fluid delivery devices. FIG. 1 illustrates an example fluid delivery device 100 from the '482 application. The fluid delivery device 100 includes an applicator 102 and a cartridge 104 that is removably positioned within the applicator 102.

FIG. 2 illustrates an example cartridge 104 from the '482 application. The cartridge 104 includes a housing 206 and a head 208 that is attached to the housing 206. The head 208 may optionally include a protective head cover 210. As shown in FIG. 3, the housing 206 is a fluid reservoir or forms a chamber 312 in which the fluid is accommodated.

The head 208 is coupled to the housing 206 to dispense the fluid from the chamber 312. Generally, the head 208 is at least temporarily in fluid communication with the chamber 312 and forms a nozzle 314 and an air entry port 316. The head 208 also includes a cap 318 and a wall 320 that are at least partially movable relative to the nozzle 314. The cap 318 stays in a closed position unless fluid is about to be or is being ejected from the nozzle 314 at which time the cap 318 transitions into an open position.

The head 208 forms a holding chamber 322 that is in fluid communication with the chamber 312 and that is positioned between the nozzle 314 and the wall 320. The wall 320 is a membrane or elastomeric wall that is “squeezable” or flexible enough to deform in response to a striking force being applied to the wall 320. When a force is applied on the wall 320, the wall 320 deforms towards the nozzle 314 thereby reducing the volume of the holding chamber 322 and forcing the fluid from the nozzle 314. Movement of the wall 320 to back to its natural state after being struck fills the holding chamber 322 with fluid from the chamber 312 to prepare for another ejection of fluid.

As shown in FIGS. 4-5, the nozzle 314 may include two slit openings 424, 426 through which fluid is dispensed from the holding chamber 322. The openings 424, 426 have a longitudinal opening width LW1 that is greater than their lateral opening length LL1. The openings 424, 426 extend through a nozzle wall 428 of the nozzle 314 from an interior nozzle surface 430 of the nozzle wall 428 to an exterior nozzle surface 432 of the nozzle wall 428. The nozzle-opening configuration shown in FIG. 4 is but one example of the nozzle-opening configurations illustrated and discussed in the '482 application.

During use of the nozzle 314 of FIG. 5, fluid F exiting from each of the two slit openings 426, 426 may coalesce into a single fluid stream FS prior to contacting the target site TS of the user (here, the user's eye). As shown in FIG. 6, because the fluid F coalesces into a single fluid stream FS, the fluid F, after reaching the eye TS, may not be uniformly distributed over a longitudinal eye width LW2 of the eye TS. Further, as shown in FIG. 6, the fluid F may be undesirably delivered to areas of the user surrounding the eye TS, requiring the user to attend to the undesirably delivered fluid F.

FIG. 7 depicts another nozzle-opening configuration illustrated in the '482 application. The nozzle 314 of FIG. 7 includes an array of openings 734. Each opening 736 of the array of openings 734 includes a substantially truncated conical shape as it extends through the nozzle wall 428. Therefore, each opening 736 has a circular entry port 738 in the interior nozzle surface 430 that is larger than a circular exit port 740 in the exterior nozzle surface 432.

The benefit of the array of openings 734 is that they are arranged in a horizontal or linear array to form an oblong shape, which typically results in the fluid streams FS having some overlap by the time they reach the eye TS and forming a continuous oval fluid footprint very similar in shape to the oval eye opening between the eyelids EL. However, because the openings 736 are evenly spaced along the nozzle wall 428, it may be difficult to predict which fluid stream FS from which opening 736 will coalesce with another fluid stream FS from another opening 736, which may result in a non-uniform distribution on the eye TS. Further, the circular shape of each of the entry and exit ports 738, 740 may take up more space along a longitudinal nozzle width LW3 of nozzle wall 428 in order to deliver a predetermined amount of fluid F than other shapes.

### SUMMARY

In an aspect, alone or in combination with any other aspect, a non-gravitational fluid delivery device is provided for delivering fluid to an eye of a user. The device comprises a nozzle having a nozzle wall. The nozzle wall has opposing interior and exterior nozzle surfaces, and a plurality of openings dispersed along a longitudinal nozzle width of the nozzle wall through which fluid is configured to be selectively delivered to the eye during use of the device. Each opening extends through the nozzle wall from a substantially rectangular entry port in the interior nozzle surface to a substantially rectangular port in the exterior nozzle surface. Each of the openings has a longitudinal opening width that is less than a lateral opening length.

In an aspect, alone or in combination with any other aspect, a nozzle for a fluid delivery device comprises a nozzle wall having opposing interior and exterior nozzle surfaces, and a plurality of openings dispersed along a longitudinal nozzle width of the nozzle wall. Each opening extends through the nozzle wall from an exit port in the interior nozzle surface to an exit port in the exterior nozzle surface. The openings are separated into opening subgroups. Each opening subgroup comprises at least one of the openings. At least one of the opening subgroups comprises at least two of the openings. A longitudinal distance between directly adjacent corresponding exit ports of a corresponding opening subgroup is less than a longitudinal distance between directly adjacent exit ports of two directly adjacent opening subgroups.

### BRIEF DESCRIPTION OF THE DRAWINGS

For a better understanding, reference may be made to the accompanying drawings, in which:

FIG. 1 is a perspective front view of a prior art non-gravitational fluid delivery device;

FIG. 2 is a perspective rear view of a component of the prior art non-gravitational fluid delivery device of FIG. 1;

FIG. 3 is a cross-sectional view of a portion of the component of FIG. 2;

FIG. 4 is a cross-sectional view of a portion of the component of FIG. 2;

FIG. 5 is a front view of a portion of the component of FIG. 2 in an example use environment;

FIG. 6 is an illustration of a target site of a user following a use of the portion of the component shown in FIG. 5;

FIG. 7 is a cross-sectional view of a portion of the component of FIG. 2, including the component in an alternate configuration;

FIG. 8 is a front view of a portion of a nozzle according to one aspect of the present invention;

FIG. 9 is a cross-sectional view of a portion of the nozzle of FIG. 8;

FIG. 10 is a front view of the nozzle of FIG. 8 in an example use environment;

FIG. 11 is an illustration of a target site of a user following a use of the nozzle of FIG. 10;

FIG. 12 is a perspective rear view of a portion of the nozzle of FIG. 8, including the nozzle in an alternate configuration;

FIG. 13 is a cross-sectional view of a portion of the nozzle of FIG. 12;

FIG. 14 cross-sectional view of a portion of the nozzle of FIG. 8 in an example use environment;

FIG. 15 is a cross-sectional view of a portion of the nozzle of FIG. 8, including the nozzle in an alternate configuration;

FIG. 16 is a cross-sectional view of a portion of the nozzle of FIG. 8, including the nozzle in an alternate configuration; and

FIG. 17 is a cross-sectional view of a portion of the nozzle of FIG. 8, including the nozzle in an alternate configuration.

### DESCRIPTION OF ASPECTS OF THE DISCLOSURE

As used herein, the term “user” can be used interchangeably to refer to an individual who prepares for, assists with, and/or performs the operation of a tool, and/or to an individual who prepares for, assists with, and/or performs a procedure.

As used herein, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, phrases such as “between X and Y” can be interpreted to include X and Y.

As used herein, the phrase “at least one of X and Y” can be interpreted to include X, Y, or a combination of X and Y. For example, if an element is described as having at least one of X and Y, the element may, at a particular time, include X, Y, or a combination of X and Y, the selection of which could vary from time to time. In contrast, the phrase “at least one of X” can be interpreted to include one or more Xs.

It will be understood that when an element is referred to as being “on,” “attached” to, “coupled” with, etc., another element, it can be directly on, attached to or coupled with the other element or intervening elements may also be present. It will also be appreciated by those of skill in the art that references to a structure or feature that is disposed “directly adjacent” another feature may have portions that overlap or underlie the adjacent feature.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as about 1 to about 6 should be considered to have specifically disclosed subranges such as about 1 to about 3, about 1 to about 4, about 1 to about 5, about 2 to about 4, about 2 to about 6, about 3 to about 6 etc., as well as individual and partial numbers within that range, for example, 1, 1.1, 2, 2.8, 3, 3.2, 4, 4.7, 4.9, 5, 5.5 and 6. This applies regardless of the breadth of the range.

The invention comprises, consists of, or consists essentially of the following features, in any combination.

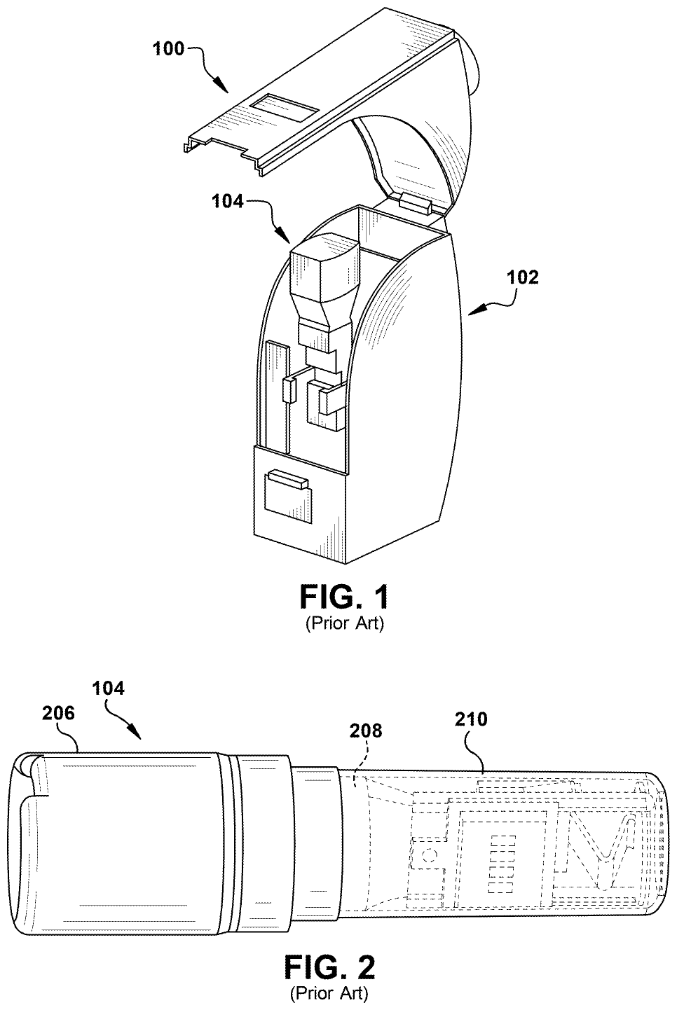

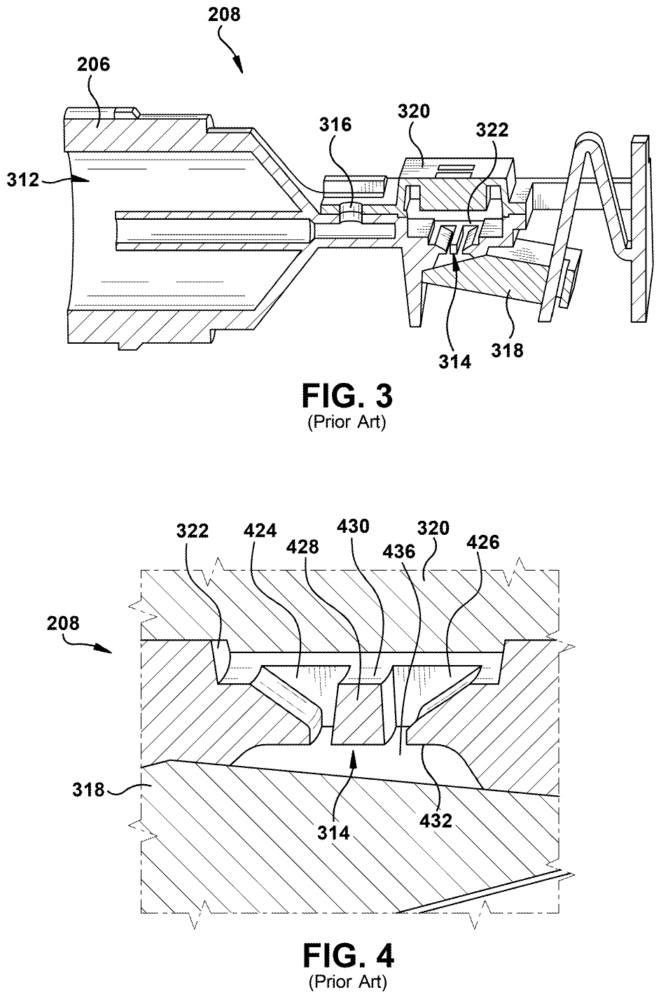

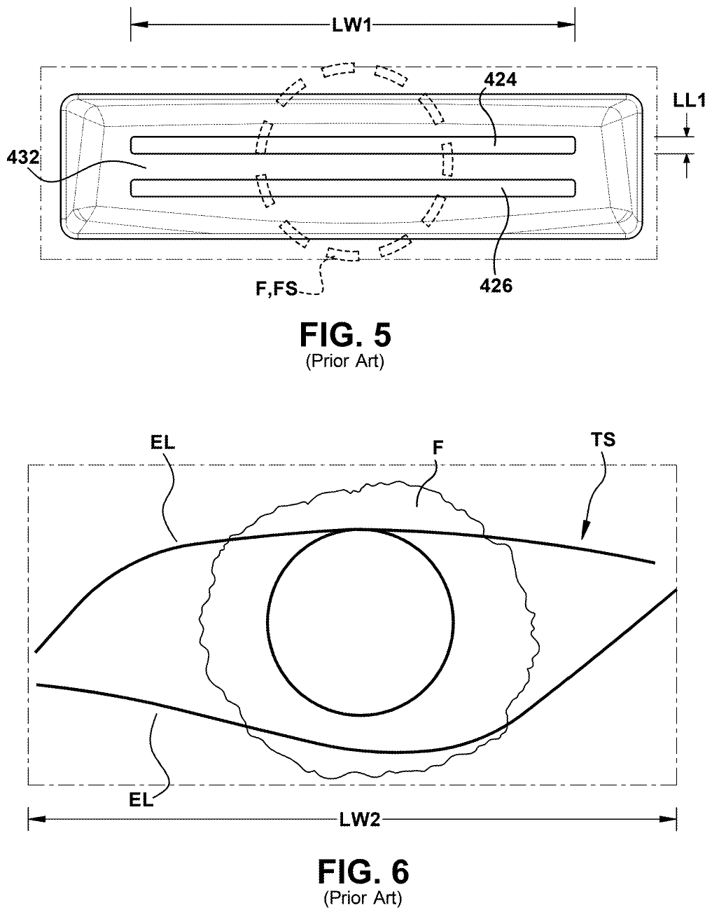

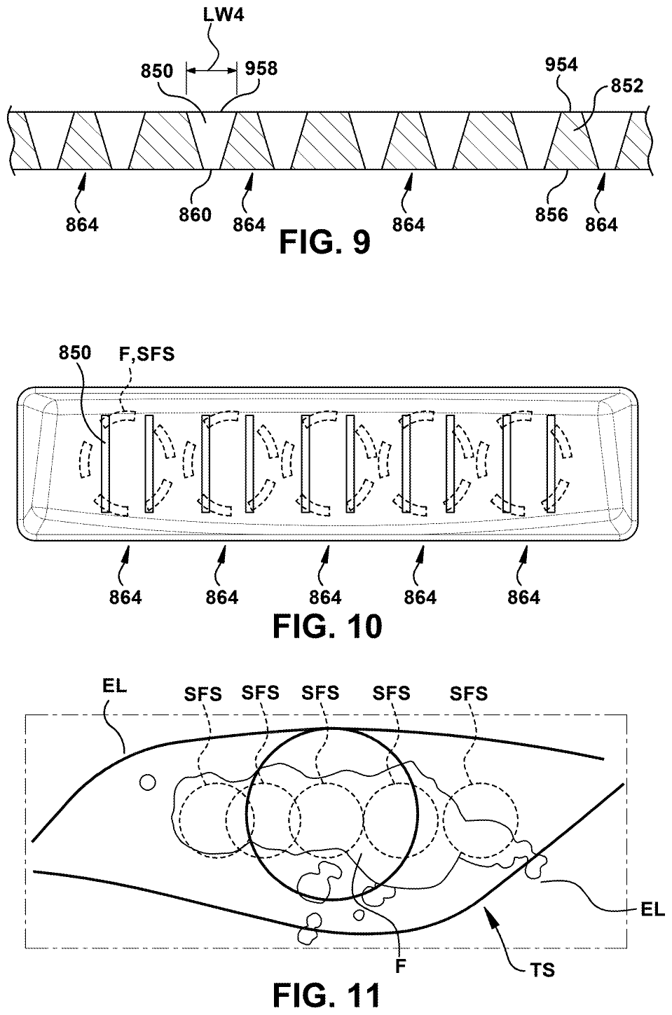

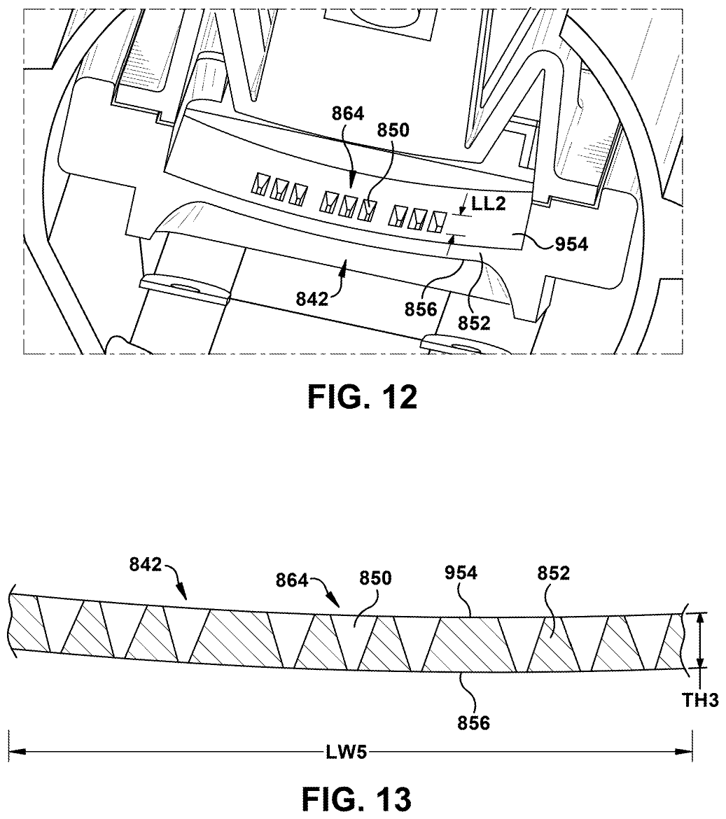

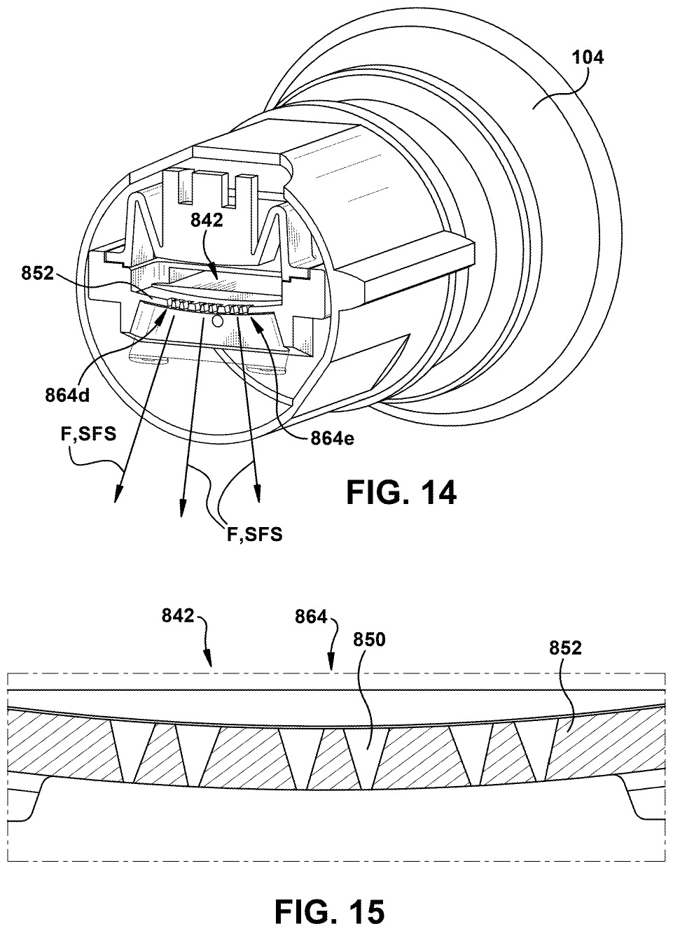

FIGS. 8-9 depict an example nozzle 842 designed in accordance with the present disclosure. For reasons of clarity, FIG. 8 depicts a front view of the example nozzle 842, which would be substantially similar to a “bottom” looking substantially “upward” view when the nozzle 842 of FIGS. 8-9 is in a similar orientation to that of FIGS. 12 and 14. The nozzle 842 and/or other teachings disclosed herein may be applicable to non-gravitational fluid delivery devices (e.g., the non-gravitational fluid delivery device(s) 100 of FIGS. 1-7 and/or any non-gravitational fluid delivery device disclosed in the '482 application), gravitational fluid delivery devices (e.g., a standard eye dropper), and/or any other fluid delivery device. In the example configuration of FIGS. 8-9, the nozzle 842 is a part of a non-gravitational fluid delivery device 844 and is formed integrally with a head 846 of a cartridge 848. The non-gravitational fluid delivery device 844, the head 846, and the cartridge 848 may substantially be, or may substantially be a modified version of, the non-gravitational fluid delivery device(s) 100, the head(s) 208, and the cartridge(s) 104 as depicted in FIGS. 1-7.

Although the nozzle 842 is described as being formed integrally with the head 846, the nozzle 842 may be formed separately from the head 846 and then attached thereto.

As shown in FIGS. 8-9, the nozzle 842 includes a plurality of openings 850 through which fluid (e.g., an ophthalmic drug and/or a viscous ophthalmic drug) is dispensed to a target site TS of a user. Although the target site TS is shown herein as being an eye of a user, the target site TS may be any other portion of the user, such as, for example, the user's nose, mouth, ear(s), limb(s), trunk, neck, and/or eye(s). Each opening 850 includes a substantially truncated pyramid shape as each opening 850 extends through a nozzle wall 852 of the nozzle 842 from an interior nozzle surface 954 of the nozzle wall 852 to an exterior nozzle surface 856 of the nozzle wall 852. Each opening 850 thus has a rectangular entry port 958 in the interior nozzle surface 954 that is larger than a rectangular exit port 860 in the exterior nozzle surface 856. The exterior nozzle surface 856 is configured to be directed substantially toward the eye TS during use. The interior nozzle surface 854, being arranged in an opposite direction from the exterior nozzle surface 856, thus is configured to be directed substantially away from the eye TS during use.

A lateral opening length LL2 of the openings 850 may be substantially the same from the entry port 958 to the exit port 860. The lateral opening length LL2, however, may be configured to at least partially inwardly taper from the entry port 958 to the exit port 860. A longitudinal opening width LW4 of the openings 850 may inwardly taper from the entry port 958 to the exit port 860. The longitudinal opening width LW4 at the entry ports 958 thus may be larger than the longitudinal opening width LW4 at the exit ports 860, which helps define the openings′ truncated pyramid shape. Although the lateral opening length LL2 of each opening 850 is shown as being greater than the longitudinal opening width LW4 of each opening 850, the openings 850 may be configured such that the lateral opening length LL2 of each opening 850 is less than the longitudinal opening width LW4 of each opening 850.

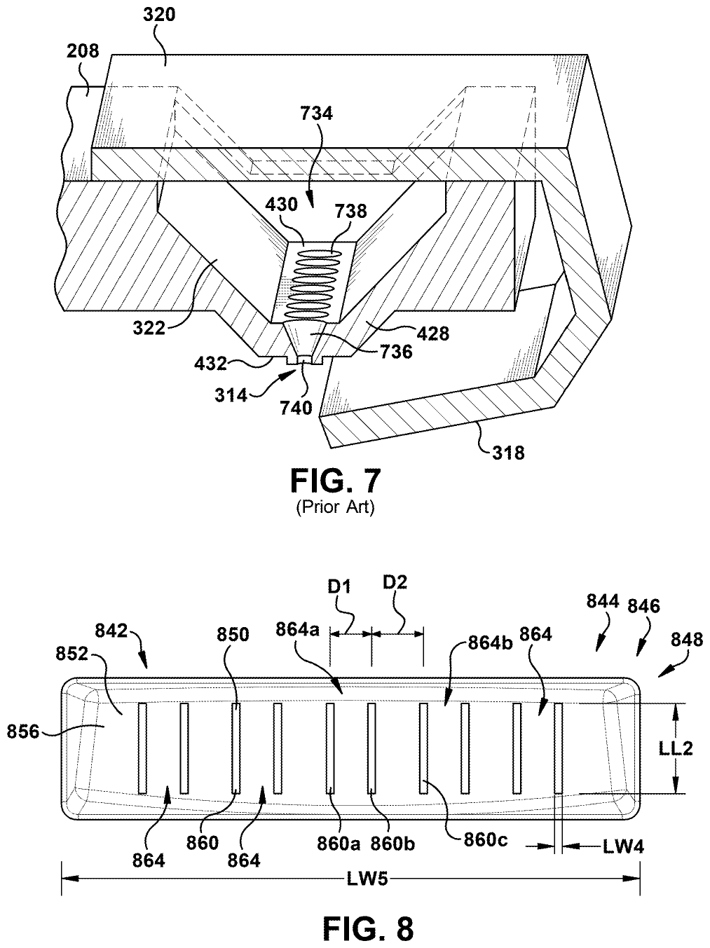

As shown in FIGS. 8-9, the openings 850 are dispersed along a longitudinal nozzle width LW5 of the nozzle wall 852. However, unlike the openings 736 shown in FIG. 7, the openings 850 shown in FIGS. 8-9 are separated into a plurality of opening subgroups 864 (as shown here, four opening subgroups 864). A longitudinal distance between directly adjacent openings 850 of an associated opening subgroup 864 is less than a longitudinal distance between directly adjacent openings 850 of two directly adjacent opening subgroups 864. For example, as shown in FIG. 8, a longitudinal distance LD1 between directly adjacent associated exit ports 860 (shown here as exit ports 860a and 860b) belonging to one opening subgroup 864 (shown here as opening subgroup 864a) is less than a longitudinal distance LD2 between directly adjacent exit ports 860 (shown here as exit ports 860b and 860c) of two directly adjacent opening subgroups 864 (shown here as opening subgroups 864a and 864b).

As shown in FIG. 10, because directly adjacent openings 850 of a single opening subgroup 864 are arranged closer to one another than they are to the openings 850 of a directly adjacent opening subgroup 864, fluid F exiting associated openings 850 of an associated opening subgroup 864 coalesce into a single corresponding subgroup fluid stream SFS in mid-air prior to contacting the user's eye TS. The distancing between each opening subgroup 864 helps prevent fluid F exiting from an opening 850 of an associated opening subgroup 864 from undesirably coalescing with the fluid F exiting from an opening 850 of a separate opening subgroup 864. Therefore, the subgroup fluid streams SFS typically remain substantially separate from one another until they reach the eye TS. As shown in FIG. 11, once the subgroup fluid streams SFS coalesce at the eye TS, the coalesced fluid F forms a substantially continuous oval fluid footprint very similar in shape to the oval eye opening between the eyelids EL. The configuration of the openings 850 of FIGS. 8-10 thus have a more predictable and predefined fluid coalescence than the openings 736 of FIG. 7, which results in a more reproducible uniform distribution of fluid F on the eye TS of the user.

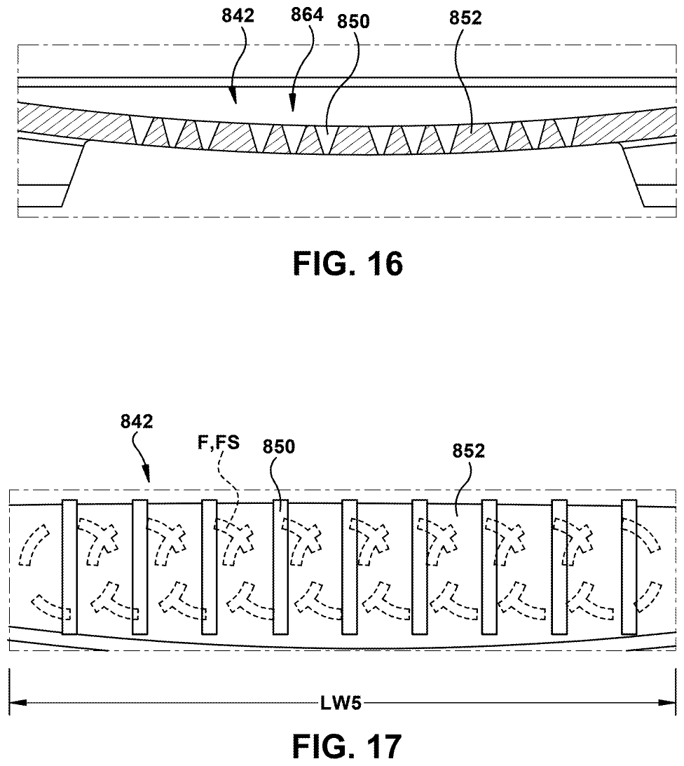

Although the nozzle 842 of FIGS. 8-9 is shown as having five opening subgroups 864 with two openings 850 in each opening subgroup 864, the nozzle 842 may have any number of opening subgroups 864 with any number of openings 850 in each opening subgroup 864. For example, as shown in FIGS. 12-14, the nozzle 842 may have three opening subgroups 864 with three openings 850 in each opening subgroup 864. In FIG. 15, the nozzle 842 is shown as having three opening subgroups 864 with two openings 850 in each opening subgroup 864. In FIG. 16, the nozzle 842 is shown as having four opening subgroups 864 with three openings 850 in each opening subgroup 864. The nozzle 842 may also be configured to have a plurality opening subgroups 864 where at least one of the opening subgroups 864 has at least two openings 850 and at least one of the opening subgroups 864 has only one opening 850. In the case of an opening subgroup 864 having only one opening 850, fluid F exiting the one opening 850 forms its own subgroup fluid stream SFS.

As shown in FIGS. 12-14, the nozzle wall 852 may be curved or arcuate along the longitudinal nozzle width LW5. In such a configuration, the interior nozzle surface 954 forms a concave-like or curved surface and the exterior nozzle surface 856 forms a convex-like or curved surface. As shown in FIG. 14, the nozzle wall 852 being curved encourages the fluid F exiting the nozzle to form a more fan-like shape after exiting. That is, fluid F exiting from the longitudinally outermost opening subgroups 864 (shown here as opening subgroups 864d, 864e) coalesce into their separate subgroup fluid streams SFS in mid-air and travel at an angle that is not perpendicular to the longitudinal direction L. The nozzle wall 852 being curved may encourage a resulting fluid footprint to be more highly elliptical or an eccentric stadium shape rather than more rounded in profile or oval shaped for larger travel distances to the eye TS.

As shown in FIGS. 12-13, the nozzle wall 852 may have a transverse wall thickness TH3 through the wall thickness separating interior from exterior of about 500 micrometers or less (e.g., about 300 to about 400 micrometers), the longitudinal opening width LW4 at the exit ports 860 may be about 75 to about 90 micrometers, and the lateral opening length LL2 of the openings 850 may be about 1 to about 2 millimeters. The perceived characteristic of a delivered dose to the eye can change from a single drop, to a several microstreams, to a finer but more chaotic spray based upon several factors including the longitudinal opening width LW4 of each opening 850, number of opening subgroups 864 and the number of openings 850 within each opening subgroup 864. By having thinner longitudinal opening widths LW4 and more openings 850, more microstreams with elongated viscous tails are produced over a larger cornea region of the eye. It is beneficial to distribute the dose over a larger region to reduce the perceived pressure of the dose. As an example, a 20 uL dose delivered at a velocity of 2.5 m/s as a single drop is of roughly 1.7 mm diameter as a higher perceived pressure than a drop that is delivered over an oblong shape 1.7 mm in one dimension and over 8 mm along the horizontal eye slit owning to the much larger surface area that the drop is spread out over. In addition to this, with viscous liquids, a microstream can form which has a viscous tail that stretches out in the direction of motion. This formation can be tuned with the longitudinal opening width LW4 and means that the drop may not instantly impact on the eye at once, but can be spread out over several microseconds. Thus, the sensation of the drop on the eye can be tailored to be barely noticeable to the user.

Although the above measurements are described in relation to the nozzle 842 configuration shown in FIGS. 12-14, such measurements may be applicable to any nozzle 842 configuration of the present disclosure.

As shown in FIG. 17, instead of being separated into subgroups 864, the openings 850 may be evenly spaced along the longitudinal nozzle width LW5 of the nozzle wall 852. The openings 850 thus may form an oblong shape, which typically results in the fluid streams FS from the separate openings 850 having some overlap by the time they reach the eye TS. Upon reaching the eye TS, the fluid F may form a continuous oval fluid footprint very similar in shape to the oval eye opening between the eyelids EL.

At least the nozzle 842 may be formed from a material and via a manufacturing process that are selected such construction of the nozzle 842 is reproducible at mass scale. For example, at least the nozzle 842 be at least partially formed from one or more plastic materials, such as, but not limited to, polyethylene (high density or otherwise) and polypropylene. To construct the nozzle 842, a mold of the nozzle 842 may first be created, e.g., via electrical discharge machining (“EDM”). One example of EDM that may be used to create the mold is wire EDM, in which a thin electrode wire that follows a precisely predetermined path is used to shape a work piece. Because the openings 850 are have a rectangular cross-section in each longitudinal plane along the transverse extension of the nozzle wall 852, taxicab geometry may be utilized in the wire EDM process to form at least a portion of the nozzle mold tooling. The nozzle 314 of FIG. 7, on the other hand, requires the use of Euclidean geometry in a wire EDM process in order to form the nozzle mold because of the circular shape of the openings 736. Use of Euclidean geometry instead of taxicab geometry when constructing a nozzle mold via wire EDM may at least partially increase the machining time and cost. After the mold of the nozzle 842 is made using EDM, nozzles 842 may be produced (e.g., mass-produced) via a molding process (e.g., injection molding). The nozzle mold could also be made, however, using any additive manufacturing, computer-aided machining, traditional manufacturing, or other fabrication processes, or combinations thereof. One of ordinary skill in the art can readily provide a suitably formed nozzle mold for a particular use situation.

Another benefit of a rectangular cross-section of the openings 850 versus the circular cross-section of the openings 736 is that for the same amount of surface area, the rectangular cross-section openings 850 may take up less space along the longitudinal nozzle width LW5 of the nozzle wall 852. More rectangular openings 850 thus may be placed along the longitudinal nozzle width LW5 of the nozzle wall 852 than circular openings 736 that have the same surface area as the rectangular openings 850. Therefore, by having rectangular openings 850, the nozzle 842 may be configured to output more fluid per a predetermined longitudinal nozzle width LW5 of the nozzle wall 852 than if the nozzle 842 had circular openings.

It should be noted that a nozzle that utilizes circular openings could be configured so that the nozzle wall has multiple rows of circular openings along the longitudinal nozzle width of the nozzle wall to achieve a similar fluid output as a nozzle that utilizes rectangular openings. However, a nozzle that has multiple rows of circular openings could be at least partially more complex to construct and less durable during use.

Another side benefit of a rectangular opening is that, for viscous liquids, more and/or smaller debris or dust may be carried away from the nozzle 842 owing to the much larger mass of liquid that can pass through a single rectangular opening 850 versus a single circular opening. While a particular sized debris could cover/clog a circular opening, the same sized debris may be less likely to cover/clog a rectangular opening 850. Thus, the rectangular openings 850 may be self-cleaning upon ejection whereas small circular openings may be more easily plugged by contamination or debris, though ideally the nozzle cap 318 keeps out debris as much as possible. Also, crusting due to loss of moisture vapor and relative humidity may be lower for a rectangular opening than a small circular opening if the nozzle cap 318 is left open for extended periods.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. In an effort to maintain clarity in the Figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the Figures. Any of the described structures and components could be integrally formed as a single unitary or monolithic piece or made up of separate sub-components, with either of these formations involving any suitable stock or bespoke components and/or any suitable material or combinations of materials. Any of the described structures and components could be disposable or reusable as desired for a particular use environment. Any component could be provided with a user-perceptible marking to indicate a material, configuration, at least one dimension, or the like pertaining to that component, the user-perceptible marking potentially aiding a user in selecting one component from an array of similar components for a particular use environment. A “predetermined” status may be determined at any time before the structures being manipulated actually reach that status, the “predetermination” being made as late as immediately before the structure achieves the predetermined status. The term “substantially” is used herein to indicate a quality that is largely, but not necessarily wholly, that which is specified—a “substantial” quality admits of the potential for some relatively minor inclusion of a non-quality item. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one aspect or configuration could be provided, singly or in combination with other structures or features, to any other aspect or configuration, as it would be impractical to describe each of the aspects and configurations discussed herein as having all of the options discussed with respect to all of the other aspects and configurations. A device or method incorporating any of these features should be understood to fall under the scope of this disclosure as determined based upon the claims below and any equivalents thereof.

Other aspects, objects, and advantages may be obtained from a study of the drawings, the disclosure, and the appended claims.

I claim:

1. A non-gravitational fluid delivery device for delivering fluid to an eye of a user, the device comprising:
a nozzle having a nozzle wall, the nozzle wall having opposing interior and exterior nozzle surfaces, and a plurality of openings dispersed along a longitudinal nozzle width of the nozzle wall through which fluid is configured to be selectively delivered to the eye during use of the device, each opening extending through the nozzle wall from a substantially rectangular entry port in the interior nozzle surface to a substantially rectangular port in the exterior nozzle surface, each of the openings having a longitudinal opening width that is less than a lateral opening length.

2. The device of claim 1, wherein the longitudinal opening widths of the openings inwardly taper from the entry ports to the exit ports.

3. The device of claim 1, wherein each opening includes a substantially truncated pyramid shape as each opening extends through the nozzle wall from the interior nozzle surface to the exterior nozzle surface.

4. The device of claim 1, wherein the nozzle wall is arcuate along the longitudinal nozzle width.

5. The device of claim 1, wherein the openings are separated into a plurality of opening subgroups, a longitudinal distance between directly adjacent associated exit ports belonging to a single opening subgroup being less than a longitudinal distance between directly adjacent exit ports of two directly adjacent opening subgroups.

6. The device of claim 5, wherein fluid exiting the openings of a corresponding opening subgroup is configured to coalesce into a single subgroup fluid stream in mid-air prior to contacting the eye.

7. The device of claim 6, wherein the subgroup fluid stream of each opening subgroup is configured to remain substantially separate from the subgroup fluid stream of another opening subgroup until the subgroup fluid streams reach the eye.

8. The device of claim 5, wherein each opening subgroup has at least two openings.

9. The device of claim 5, wherein the nozzle wall is arcuate along the longitudinal nozzle width such that fluid exiting from a longitudinally outermost subgroup coalesces into a subgroup fluid stream in mid-air and travels at a non-perpendicular angle to a longitudinal direction.

10. The device of claim 1, wherein the nozzle wall has a transverse wall thickness of about 500 micrometers or less.

11. The device of claim 10, wherein the longitudinal opening width of each opening at the exit port is about 75 to about 90 micrometers, the lateral opening length of each opening at the exit port being about 1 to about 2 millimeters.

12. The device of claim 1, wherein at least the nozzle is formed from at least one of polyethylene and polypropylene.

13. A nozzle for a fluid delivery device, the nozzle comprising:
a nozzle wall having opposing interior and exterior nozzle surfaces, and a plurality of openings dispersed along a longitudinal nozzle width of the nozzle wall, each opening extending through the nozzle wall from an exit port in the interior nozzle surface to an exit port in the exterior nozzle surface,
the openings being separated into opening subgroups, each opening subgroup comprising at least one of the openings, at least one of the opening subgroups comprising at least two of the openings,
a longitudinal distance between directly adjacent corresponding exit ports of a corresponding opening subgroup being less than a longitudinal distance between directly adjacent exit ports of two directly adjacent opening subgroups.

14. The nozzle of claim 13, wherein the entry and exit ports of at least one of the openings are substantially rectangular.

15. The nozzle of claim 14, wherein at least one of the openings has a longitudinal opening width that is less than a lateral opening length.

16. The nozzle of claim 13, wherein the entry and exit ports of each of the openings are substantially rectangular.

17. The device of claim 16, wherein each of the openings have a longitudinal opening width that inwardly tapers from the entry ports to the exit ports such that each opening has a substantially truncated pyramid shape as each opening extends through the nozzle wall from the interior nozzle surface to the exterior nozzle surface.

18. The device of claim 13, wherein fluid exiting the openings of a corresponding opening subgroup is configured to coalesce into a single subgroup fluid stream in mid-air prior to contacting the eye.

19. The device of claim 18, wherein the subgroup fluid stream of each opening subgroup is configured to not substantially coalesce with the subgroup fluid stream of another opening subgroup until the subgroup fluid streams reach the eye.

20. The device of claim 13, wherein the nozzle wall is arcuate along the longitudinal nozzle width such that fluid exiting from a longitudinally outermost subgroup coalesces into a subgroup fluid stream in mid-air and travels at a non-perpendicular angle to a longitudinal direction.
